(19) United States
(12) Patent Application Publication
(10) Pub. No.: US 2011/0117144 A1
(43) Pub. Date: May 19, 2011

(54) Topical cosmetic preparation containing elastogenesis inducing substances associated with a systemic absorption retardant

(76) Inventors: Filiberto Palmiro Zadini, Northridge, CA (US); Giorgio Cesare Zadini, Santa Rosa Valley, CA (US)

(21) Appl. No.: 12/592,032

(22) Filed: Nov. 18, 2009

(51) Int. Cl. A61K 8/97; A61K 8/34; A61K 8/35; A61K 8/02; A61K 8/73; A61Q 19/00

(52) U.S. Cl. 424/401; 424/725; 514/730; 514/675; 514/724; 514/58

(57) ABSTRACT

A cosmetic/dermatological topical preparation such as a cream or ointment or lotion containing elastogenesis inducing substances such as a dill extract or Ethocyn associated with a systemic absorption retardant such as a mixture of Benzyl Alcohol, Acetone and Isopropanol. Such a formulation achieves high local tissue concentration of the elastogenesis inducing substances in vicinity to the site of application, via minimization of systemic absorption of the elastogenesis inducing substances and at the same time via promotion of their intradermal absorption across the epidermal barrier. As a result of the use of such formulation an increased elastogenesis activity is expected to occur in the dermis, ultimately resulting in the neo-formation of an elastin network in the adult skin. The use of other percutaneous chemical transdermal enhancers such as liposomes and cyclodextrins to be added to the cosmetical preparation or the use of physical penetration enhancers such as Micro-Dermabrasion or Iontophoresis, are also disclosed, to favor penetration of the cosmetic preparation into the skin.

### RELATED CASES

This Application claims priority on U.S. Provisional Application No. 61/199,348 filed Nov. 18, 2008; the contents of all of the foregoing are hereby incorporated by reference herein in their entireties.

### FIELD OF INVENTION

This application relates to cosmetic/dermatological topical preparations containing an elastogenesis enhancer/inducer associated with a systemic absorption retardant.

### BACKGROUND OF THE INVENTION

Elastin fibers are believed to be responsible for the elasticity of the skin. The process of aging of the skin is associated in part with gradual loss of elasticity resulting from deficiency of elastic fibers replacement in adult life. A number of enzymes of the lysyl oxidase family play an important role in the formation of elastic fibers. In particular, the enzymes lysyl oxidase (LOX) and lysyl oxidase-like (LOXL) are responsible for elastin cross-linking. It has been shown recently that LOXL is essential for the elastic fibers homeostasis and for their maintenance at adult age. It has been shown that LOXL plays a very important role not only in elastic fibers formation but also in their renewal. LOXL appears to be associated with forming elastic fibers in the human skin, as reported by Cenizo V, Andre´ V, Reymermier C, Sommer P, Damour O, Perrier E. in the article LOXL as a target to increase the elastin content in adult skin: a dill extract induces the LOXL gene expression, Exp Dermatol 2006: 15: 574-581. According to the Authors, enzymes lysyl oxidase (LOX) and lysyl oxidase-like (LOXL), in particular LOXL, can be considered as a new target to reinduce elastogenesis: its stimulation by certain substances is correlated with increased elastin detection, which suggests an increase in elastogenesis efficiency. Specific substances, which include a few phytochemicals, have shown the capability of re-inducing elastogenesis. Such substances are: dill, currant, cardamon, black radish, small holly, cinnamon, lactic bacteria-based fermentations, oats, potato, silk, Asea foetida gum, ethyl hexenoate and its derivatives, methyl butyrate and its derivatives, and ethyl decadienoate and its derivatives. In particular, a dill extract has been shown to be the most effective in re-inducing elastogenesis in adult skin when applied topically. Another elastogenesis inducer substance disclosed in this Application is Ethocyn, its chemical name being ethoxyhexyl-bicyclooctanone. Ethocyn is claimed to stimulate the synthesis of elastin in the skin.

In normal situations, however, once applied to the intact adult skin and vehicled into the dermis and dermal layer in their dermatological preparation after crossing the epidermal barrier, the elastogenesis inducers are promptly absorbed from the dermal layer into the systemic circulation. This rapid systemic absorption into the systemic circulation from the dermal layer, significantly limits the exposure of the of the LOX and LOXL to the elastogenesis inducers, with the effect of thwarting the overall effectiveness of elastogenesis induced by the dill extract and by the other compounds. Indeed in absence of ingredients named systemic absorption retardants, the elastogenesis inducers/enhancers do not station in the dermis and subcutaneous layers long enough in sufficient concentration to effectively induce elastogenesis in the skin. The elastogenesis inducers/enhancers compounds can hardly accumulate in the dermis to function as depot drug delivery system. The addition of a dermal layer absorption retardant capable of hindering the systemic absorption of the elastogenesis inducers can prolong the exposure of the LOX and LOXL to elastogenesis inducers so as to maximize the topical elastogenesis effect induced by the elastogenesis inducers.

In addition to subcutaneous absorption retardants, the Applicants also propose the use of various percutaneous delivery enhancers to favor the penetration of the elastogenesis inducers into the dermis and dermal layer through the skin barrier represented by the epidermal stratum corneum.

### BRIEF SUMMARY OF THE INVENTION

With the present invention Applicants propose a topical cosmetic/dermatological preparation such as a cream or ointment or lotion or the likes, containing an elastogenesis enhancer/inducer associated with a systemic absorption retardant. Applicants disclose the use of agents which retard the absorption of the active ingredients from the dermal layer allowing the active ingredients to act longer at a greater local concentration. Applicants also discloses the use of percutaneous delivery enhancers that can be added to the elastogenesis inducers/systemic absorption retardants combination so as to enhance passage of elastogenesis inducers into the dermis and dermal layer.

More specifically, Applicants disclose the addition/association of a mixture of three components, Benzyl Alcohol, Acetone and Isopropanol to the cosmetic cream containing the elastogenesis inducer. Although in “Delivery of erythromycin to dermal layers in rats by means of a trans-phase delivery system” by Peng L, Nimni M E, J Pharm Pharmacol. 1999 October; 51(10):1135-41, the mixture of Benzyl Alcohol, Acetone and Isopropanol has been proposed in association with the topical absorption of erythromycin, the Applicants postulate its usefulness also with elastogenesis inducers. Remarkably, in the case of erythromycin, the addition of Benzyl Alcohol, Acetone and Isopropanol mixture not only works as a subcutaneous absorption retardant but also as a transdermal permeability enhancer. The Applicants assert that the mixture of Benzyl Alcohol, Acetone and Isopropanol hinders systemic absorption of the elastogenesis inducer, which results in significant and persistent local accumulation in the dermis and dermal layer of the elastogenesis inducer being transdermally delivered across the stratum corneum of the epidermis, with overall maximization of the local effect of the elastogenesis inducer. The Benzyl Alcohol, Acetone and Isopropanol mixture represents an ideal addition to the cosmetic preparation aimed at rebuilding the elastic fibers network in the skin as not only it acts as a subcutaneous absorption retardant delaying the clearance of the elastogenesis inducers from the dermal layer, but also acts as a percutaneous transdermal delivery enhancer, promoting conveyance of the elastogenesis inducer into the dermis and dermal layer with an overall effect of accumulation of elastogenesis inducer precisely where it is required to be, with a resulting maximization of the elastogenesis effect.

Applicants also propose the use of other transdermal delivery enhancers including physical transdermal enhancers.

### OBJECTS OF THE INVENTION

It is an object of the present invention to provide a cosmetic preparation capable of enhancing availability of the ingredients capable of re-induce elastogenesis in the skin, ultimately improving the skin texture and appearance.

It is an object of the present invention to provide a cosmetic preparation with a mixture of ingredients capable of favoring local accumulation of the active cosmetic ingredients with maximization of the local effect of the active ingredients.

It is an object of the present invention to provide a cosmetic preparation with a mixture of ingredients capable of favoring local accumulation of the active cosmetic ingredients via a decrease of clearance of such active cosmetic ingredients from the dermal layer and via enhancement of percutaneous transdermal delivery into the dermal layer to maximize accumulation of active cosmetic ingredients in the dermal layer wherein

### FIGURES

FIG. 1 shows a Liposome with a payload containing elastogenesis inducing substances associated with an absorption retardant.

FIG. 2 shows a Cyclodextrin with a payload containing elastogenesis inducing substances associated with an absorption retardant.

### SPECIFICATIONS

The preferred embodiment of this invention consists of a dermatological/cosmetic topical preparation such as a cream or ointment containing an elastogenesis inducer or inducers such as dill extract at various concentrations such as 2% or more or other compounds having the same characteristic of inducing elastogenesis in the skin such as currant, cardamon, black radish, small holly, cinnamon, lactic bacteria-based fermentations, oats, potato, silk, Asea foetida gum, ethyl hexenoate and its derivatives, methyl butyrate and its derivatives, ethyl decadienoate and its derivatives, said elastogenesis inducing ingredients being associated with a local intradermal absorption retardant or retardants.

Another elastogenesis inducer to be used in association with dermal layer absorption retardant is Ethocyn, chemical name ethoxyhexyl-bicyclooctanone. As pointed out above, it is claimed to stimulate the synthesis of elastin in the skin. As per its mechanism of action, Ethocyn is claimed to block the action of the androgenic sex hormone DHT, i.e. dehydrotestosterone. It is a well-established fact that DHT levels increase with age. Ethocyn makers claim that “age-related decline of elastin synthesis is caused mainly by high levels of DHT. Therefore, topical application of Ethocyn presumably blocks DHT in the skin and thereby stimulates elastin synthesis.”

Applicants propose the used of Ethocin with a systemic absorption retardant, to delay the systemic absorption of Ethocyn from the dermal layer and maximize its topical effect on the skin. Maximization of its local effect, as in the case of any other elastin fibers network neoformation inducer, is predicted by Applicants to be greater than when the elastin synthesis inducer is used alone.

The intradermal systemic retardants disclosed in this Applications acts also as a skin penetration enhancer.

The purpose for associating in the present application elastogenesis inducers substance or substances alone or in association and an intradermal absorption retardant mixture is to deliver the elastogenesis inducer locally into the dermis minimizing systemic absorption from the dermal layer so as to maximize intradermal and subcutaneous accumulation in order to achieve maximization of local elastogenic effect.

Indeed, in situations in which an intradermal absorption retardant is not used, the dermal microvasculature absorbs the drug being delivered rapidly placing it into the systemic circulation before it ever reaches the targeted dermal regions.

In the cited study conducted at USC, a mixture of Benzyl Alcohol, Acetone and Isopropanol has resulted not only with a promotion of intradermal delivery of the erythromycin, but also with a significant and persistent local accumulation of erythromycin in the dermal layer, while, on the contrary, the use of a standard hydrophilic transdermal carrier, such as propylene glycol, has resulted with a rapid systemic absorption of the transdermally delivered erythromycin and with a very negligible and short lived accumulation of the erythromycin in the dermal layer.

Applicants assert that such mixture of Benzyl Alcohol, Acetone and Isopropanol combined with the elastogenesis inducer will promote transport of the elastogenesis inducer across the epidermal barrier while at the same time such a mixture will minimize systemic absorption with a resulting significant and persistent accumulation of the elastogenesis inducer in the dermis and in the subcutaneous tissue. Such a formulation has the potential for achieving high local tissue concentration in the vicinity of the site of application, and, consequently, it has the potential for maximizing the local pharmacological effects of the elastogenesis inducing substances in the dermis and dermal layer in the proximity of the site of application, and ultimately of recreating skin elasticity at the site of application. The respective concentration of Benzyl Alcohol, Acetone and Isopropanol varies, however, the recommended concentration is: benzyl alcohol (10%), acetone (40%), isopropanol (50%). Applicants propose the use of the mixture of Benzyl Alcohol, Acetone and Isopropanol in which at least two of the three components are present.

Other skin penetration enhancers which facilitate transport of the active principles thru the skin into the target tissue can be added to the dermatological/cosmetic preparation containing the elastogenesis inducing ingredients and an intradermal absorption retardant such as the Benzyl Alcohol, Acetone and Isopropanol mixture.

The skin permeability enhancers that can be used include Percutaneous Chemical Enhancers and Percutaneous Physical Enhancers.

The Percutaneous Chemical Enhancers which can be added to the compound can be classified as: Cyclodextrins, Liposomes, Ethosomes, Sulfoxides, Alcohols, Fatty acids, Fatty acid esters, Polyols, Amides Surfactants, Terpene, Alkanones Organic acids.

Specifically Percutaneous Chemical Enhancers which can be used are:

Ethanol, Glyceryl monoethyl ether, Monoglycerides, Isopropylmyristate, Lauryl alcohol (also, lauric acid, lauryl lactate), Terpinol, Menthol, D-limonene, Beta-cyclodextrin, DMSO (dimethyl sulfoxide), Polysorbates, Fatty acids e.g. oleic, N-methylpyrrolidone, Polyglycosylated glycerides, 1-Dodecylaza cycloheptan-2-one known as Azone®, Cyclopentadecalactone known as CPE-215®, Alkyl-2-(N,N-disubstituted amino)-alkanoate ester, known as NexAct®, 2-(n-nonyl)-1,3-dioxolane known as SEPA®.

In this specific case it is logic to deduct that local accumulation of the elastogenesis inducing substances in the dermis will enhance local effects of the active ingredients due to the fact that uptake of the therapeutic substance into the systemic circulation is delayed.

In addition to chemical transdermal permeability enhancers, physical transdermal enhancers can be used. Physical Enhancers which can be used are Iontophoresis, Electroporation, Sonophoresis, Thermal Poration and in general physically/chemically induced heat, Microneedles, Dermabrasion. Topical delivery into the dermal layers of elastogenesis inducing substances can also be enhanced after Microdermabrasion. Microdermabrasion can be used alone or in combination with Percutaneous Chemical Enhancers, such as the Percutaneous Chemical Enhancers described above and/or with Physical Enhancers, such as the Physical Enhancers described above.

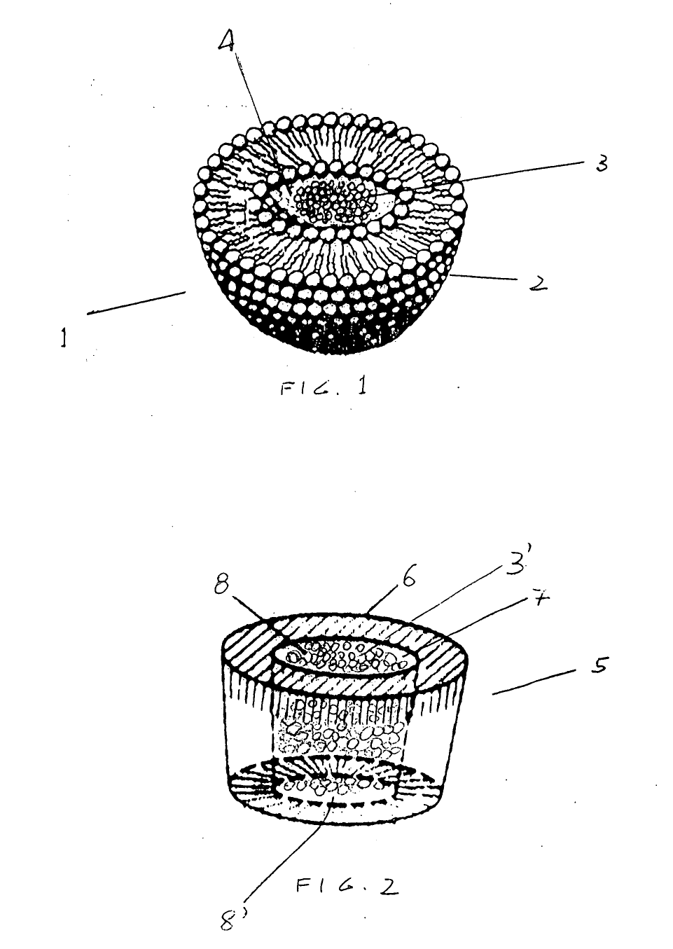

FIG. 1 shows liposome vesicle 1 in cross section.

As shown in FIG. 1, liposome vesicle 1 is of spherical shape with multilayer phospholipids membrane wall 2 delimiting inner cavity 4 for payload 3.

Payload 3 can be any of the above mentioned elastogenesis inducers/enhancers such as dill extract or currant, cardamon, black radish, small holly, cinnamon, lactic bacteria-based fermentations, oats, potato, silk, Asea foetida gum, ethyl hexenoate and its derivatives, methyl butyrate and its derivatives, ethyl decadienoate and its derivatives, alone or in a mixture or Ethocyn said elastogenesis inducing ingredients being associated within the Liposome vesicle with a Benzyl Alcohol, Acetone and Isopropanol mixture in the concentration disclosed above.

FIG. 2 shows cyclodextrin unit 5 generally of toroid shape having polysaccharide wall 6 delimiting cavity 7 for payload 3′. Toroid shaped cyclodextrin 5 is formed with upper larger opening 8 and lower smaller opening 8A. Payload 3′ housed within cavity 7, is the same as for Liposome 1.

What we claim is:

1. A topical dermatological preparation applied onto a skin of a patient to restore elasticity to the skin, the skin including an epidermal and a dermal layer, the dermal layer also known as dermis, said topical dermatological preparation comprising;
an elastogenesis inducer substance and
a systemic absorption retardant to delay absorption of said elastogenesis-inducer substance into a systemic circulation to minimize systemic absorption from the dermal layer of the skin so as to maximize intradermal accumulation of said elastogenesis-inducer substance in the dermis in order to achieve maximization of local elastogenic effect.

2. The elastogenesis inducer substance of claim 1 being a phytochemical with biological properties capable of inducing elastogenesis.

3. The elastogenesis inducer substance of claim 2 being a dill extract.

4. The dill extract of claim 3 being at a concentration of 2%.

5. The dill extract of claim 3 being at a concentration greater than 2%.

6. The elastogenesis inducer substance of claim 2 being Ethocyn.

7. The systemic absorption retardant retarding the absorption of the elastogenesis-inducer substance of claim 1 into the systemic circulation is a mixture of Benzyl Alcohol, Acetone and Isopropanol wherein at least two of three components are present.

8. The systemic absorption retardant of claim 7 retarding the absorption of the elastogenesis-inducer substance into the systemic circulation is also used as skin permeability enhancer.

9. The topical dermatological preparation of claim 1 wherein said elastogenesis inducer substance being used in association with said systemic absorption retardant is also used in association with a percutaneous permeability enhancer.

10. The topical dermatological preparation of claim 9 wherein said percutaneous permeability enhancer is a chemical permeability enhancer.

11. The topical dermatological preparation of claim 10, wherein said percutaneous chemical permeability enhancer is a liposome.

12. The topical dermatological preparation of claim 10, wherein said percutaneous chemical permeability enhancer is a cyclodextrin.

13. The topical dermatological preparation of claim 10, wherein said percutaneous chemical permeability enhancer is a chemically induced heat.

14. The topical dermatological preparation of claim 9 wherein said percutaneous permeability enhancer is a physical permeability enhancer.

15. The topical dermatological preparation of claim 14, wherein said percutaneous physical permeability enhancer is a dermabrasion.

16. The topical dermatological preparation of claim 14, wherein said percutaneous physical permeability enhancer is a microdermabrasion.

17. The topical dermatological preparation of claim 14, wherein said percutaneous physical permeability enhancer is a iontophoresis.

18. The topical dermatological preparation of claim 14, wherein said percutaneous physical permeability enhancer is a microneedles apparatus.

19. The topical dermatological preparation of claim 14, wherein said percutaneous physical permeability enhancer is electroporation,

20. The topical dermatological preparation of claim 14, wherein said percutaneous physical permeability enhancer is a sonophoresis,

21. The topical dermatological preparation of claim 14, wherein said percutaneous physical permeability enhancer is a physically induced heat.
